(19) United States
(12) Patent Application Publication
(10) Pub. No.: US 2026/0004905 A1
(43) Pub. Date: Jan. 1, 2026

(54) ORAL TRACKER

(71) Applicant: Carolina Medical Electronics Inc., East Bend, NC (US)

(72) Inventor: Bradley David Brown, Dobson, NC (US)

(21) Appl. No.: 19/252,040

(22) Filed: Jun. 27, 2025

Related U.S. Application Data
(60) Provisional application No. 63/665,306, filed on Jun. 28, 2024.

(51) Int. Cl. G16H 20/00; G16H 10/20; G16H 40/67

(52) U.S. Cl. CPC G16H 20/00; G16H 10/20; G16H 40/67

(57) ABSTRACT

An oral consumable tracker device for tracking use and consumption of oral consumables. The oral consumable tracker device is configured for research studies and medical purposes to monitor and track behavior corresponding to oral consumable (e.g., tobacco pouches). The oral consumable tracker device includes means for monitoring access and removal of oral consumables.

### CROSS-REFERENCE TO RELATED APPLICATIONS

This application claims priority to and the benefit of U.S. Provisional Patent Application No. 63/665,306, filed on Jun. 28, 2024, the entire contents of which is herein incorporated by reference in its entirety.

### BACKGROUND

Product testing and corresponding data is critical for companies to properly analyze how consumers use products and to ensure proper adherence during research studies. Companies need to capture data and track product usage for a given product for research purposes and/or data submission to a regulatory agency. When the product is an oral product, it is difficult to accurately capture data and user behavior. Currently there is nothing on the market available for companies to use for oral product testing studies to capture the data required.

### FIELD OF THE DISCLOSURE

The present disclosure generally relates to product tracking, more specifically to tracking use of oral consumables during clinical and research studies.

### DESCRIPTION OF RELATED ART

Tracking behavior during clinical trials and research studies can be difficult due to manual reporting and the potential for human error. These problems can lead to inaccuracies and biases, potential data tampering, and affect the reliability of studies. Therefore, there is a need to address the aforementioned problems for capturing, monitoring, and analyzing behavior corresponding to use and consumption of oral products.

### BRIEF SUMMARY

In at least one aspect of the present disclosure, an oral consumable tracking system is disclosed. The oral consumable tracking system can include at least one remote device and at least one oral consumable tracker device. The oral consumable tracker device can include a housing with at least two interior compartments, a base, and a lid, an interior layer with a slidable door, and a user interface. The oral consumable tracker device is usable for tracking the use of products including oral tobacco pouches, orbs, and other consumables. For example, and without limitation, a first compartment may be used for storage of an oral consumable (e.g., medication). The oral consumable tracker device is configured to detect when at least a portion of an oral consumable has been removed. The second compartment may be used for waste management and is operable to detect when packaging of a consumable is positioned in the second compartment. For example, and without limitation, the oral consumable tracker device can include at least one sensor for monitoring each compartment. The oral consumable tracker device can receive and request user input via the user interface. The user input may include survey questions, indication of usage of the oral consumable tracker device, and indication of ending the usage of the oral consumable tracker device. The oral consumable tracker device is configured to upload user input and activity data to at least one remote server including a remote database.

In at least one aspect of the present disclose, a method of tracking consumption of oral consumables is disclosed. The method includes (1) receiving user input via a user interface indicating that a user is beginning consumption of an oral consumable, (2) detecting removal of a lid of a housing of an oral consumable tracker device, (3) detecting a change in an interior component of the oral consumable tracker device corresponding to an oral consumable, (4) detecting remaining oral consumable (e.g., packaging) via a second interior compartment of the oral consumable tracker device, (5) detecting placement of the lid back on the housing, and (6) requesting user input to end a session. The method may further include automatically updating a database based on the detected behavior corresponding to the consumable. In at least one aspect of the present disclosure, the second compartment can be used for a second oral consumable.

In at least one aspect of the disclosure, an oral consumable tracker device is disclosed. The oral tracker device includes a top shell, a bottom shell, at least one power supply, a printed circuit board assembly including at least one processor and at least one antenna, and at least one user interface. The bottom shell of the oral consumable tracker device includes at least two compartments. Each compartment of the at least two compartments can track and monitor the placement and use of an oral consumable inserted and removed from each compartment. In at least one aspect of the present disclosure, the top shell and/or the bottom shell may include at least one sensor for tracking consumption of the oral consumable. In at least one aspect of the present disclosure, the oral consumable tracker device includes at least one slidable layer positioned between the top shell and the bottom shell. The slidable layer may include a door designed to cover one or both of the at least two compartments. The oral consumable tracker device can recognize when the at least one slidable layer is positioned over a compartment.

### BRIEF DESCRIPTION OF THE SEVERAL VIEWS OF THE DRAWINGS

The embodiments illustrated, described, and discussed herein are illustrative of the present disclosure. As these embodiments of the present disclosure are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present disclosure, and through which these teachings have advanced the art, are considered to be within the spirit and scope of the present disclosure. Hence, these descriptions and drawings should not be considered in a limiting sense, as it is understood that the present disclosure is in no way limited to only the embodiments illustrated.

FIG. 1 illustrates a top perspective view of an oral consumable tracker device according to at least one aspect of the present disclosure.

FIG. 2 illustrates an exploded view of an oral consumable tracker device according to at least one aspect of the present disclosure.

FIG. 3 illustrates a bottom shell of a housing of an oral consumable tracker device according to at least one aspect of the present disclosure.

FIG. 4 illustrates a top view of a base of an oral consumable tracker device according to at least one aspect of the present disclosure.

FIG. 5 illustrates a perspective view of a base of an oral consumable tracker device according to at least one aspect of the present disclosure.

FIG. 6 illustrates a schematic diagram of an oral consumable tracking system according to at least one aspect of the present disclosure.

FIG. 7 illustrates a schematic diagram of an oral consumable tracking system according to at least one aspect of the present disclosure.

FIG. 8 illustrates a schematic diagram of a remote server of an oral consumable tracking system according to at least one aspect of the present disclosure.

FIG. 9 illustrates a schematic diagram of a computer of an oral consumable tracking system according to at least one aspect of the present disclosure.

FIG. 10 illustrates a schematic diagram of a mobile device of an oral consumable tracking system according to at least one aspect of the present disclosure.

FIG. 11 illustrates a schematic diagram of an Internet-of-Things (IoT) of an oral consumable tracking system according to at least one aspect of the present disclosure.

### DETAILED DESCRIPTION

For the purposes of promoting an understanding of the present disclosure, reference will be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “a composite” means at least one composite and can include more than one composite.

Throughout the specification, the terms “about” and/or “approximately” may be used in conjunction with numerical values and/or ranges. The term “about” is understood to mean those values near to a recited value. For example, “about 40 [units]” may mean within +/−25% of 40 (e.g., from 30 to 50), within +/−20%, +/−15%, +/−10%, +/−9%, +/−8%, +/−7%, +/−6%, +/−5%, +/−4%, +/−3%, +/−2%, +/−1%, less than +/−1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

As used herein, the verb “comprise” as is used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only”, and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

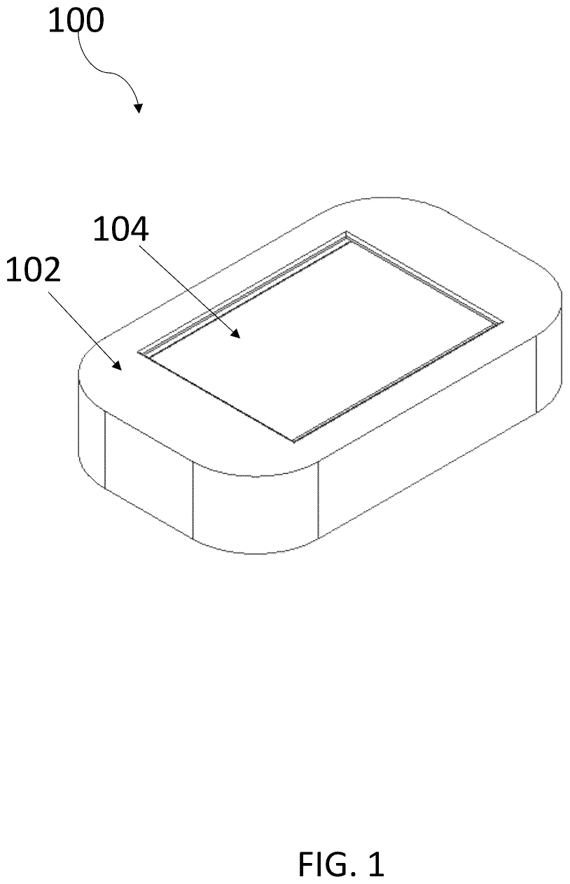

FIG. 1 illustrates an oral consumable tracker device according to at least one aspect of the present disclosure. The oral consumable tracker device 100 includes a top shell 102, a bottom shell, at least one power supply component, a printed circuit board assembly including at least one processor, means for monitoring use of oral consumables, and a user interface 104. The oral consumable tracker device can be configured for network communication with at least one remote device. The means for monitoring use of oral consumables can include at least one sensor. The at least one power supply component can include a battery. The battery can be rechargeable or non-rechargeable. Via the user interface, the oral consumable tracker device can receive user input. The user input may include response to survey questions, indicating a start of use of an oral consumable, and indicating an end of use of an oral consumable.

The at least one sensor can include a magnetic field sensor, a linear sensor, a switch sensor, a Hall Effect sensor, a push switch, a proximity sensor, a weight sensor, an image sensor, or a motion sensor. The at least one sensor can be positioned on or in the bottom shell and/or the top shell. For further example, in at least one aspect of the present disclosure, the oral consumable tracker device can monitor a change in a weight and/or movement of an oral consumable in each compartment. The means for monitoring use of oral consumables can compare a weight before a user removes an oral consumable to a weight when a user returns the oral consumable and/or the oral consumable packaging. The oral consumable tracker device may generate an alert if the weight does not change or is above or below an expected threshold. If the determined weight is within a predetermined threshold, the oral consumable tracker device can automatically update user information to a remote database.

In at least one aspect of the present disclosure, the oral consumable tracker device includes a controllable layer positioned between the top shell and the bottom shell. In at least one aspect of the present disclosure, the top shell is removably attached to the bottom shell. For example, and without limitation, the bottom shell includes at least one feature for receiving the top shell. The feature can include a channel, a slot, or a similar feature for receiving the top shell. In at least one aspect of the present disclosure, the top shell can nestle in the bottom shell. In at least one aspect of the present disclosure, the top shell can snap onto the bottom shell.

In at least one aspect of the present disclosure, the controllable layer may include a slidable layer that can cover one or more compartments of the at least two compartments. The controllable layer may be manually controlled, controlled by the at least one processor, and/or remotely controlled. For example, and without limitation, the controllable layer may include a slidable door that can be physically moved by a user. In at least one aspect of the present disclosure, the at least one processor can control the position of the controllable layer. For example, and without limitation, the at least one processor can detect that a user is beginning a session (e.g. user input via the user interface), and, in response, move the controllable layer to uncover a compartment that includes an oral consumable. For further example, and without limitation, the at least one processor can detect when at least one consumable is removed from a compartment, and, in response, move the at least one controllable layer to cover the compartment corresponding to the removed oral consumable. Advantageously, this helps improve accuracy during studies by limiting access to oral consumables. In at least one aspect of the present disclosure, the oral consumer trackable device can receive a command from at least one remote device corresponding to the position of the controllable layer.

The processor manages the overall operations of the oral consumable tracker device. The processor is any controller, microcontroller, or microprocessor that is capable of processing program instructions. In one embodiment, the oral consumable tracker device includes at least one antenna, which enables the oral consumable tracker device to send information (e.g., oral consumable consumption, battery level) to at least one remote device (e.g., smartphone, tablet, laptop computer) and/or receive information (e.g., user survey questions) from at least one remote device. The at least one antenna provides wireless communication, standards-based or non-standards-based, by way of example and not limitation, radiofrequency (RF), BLUETOOTH, ZIGBEE, NEAR FIELD COMMUNICATION (NFC), or other similar communication methods.

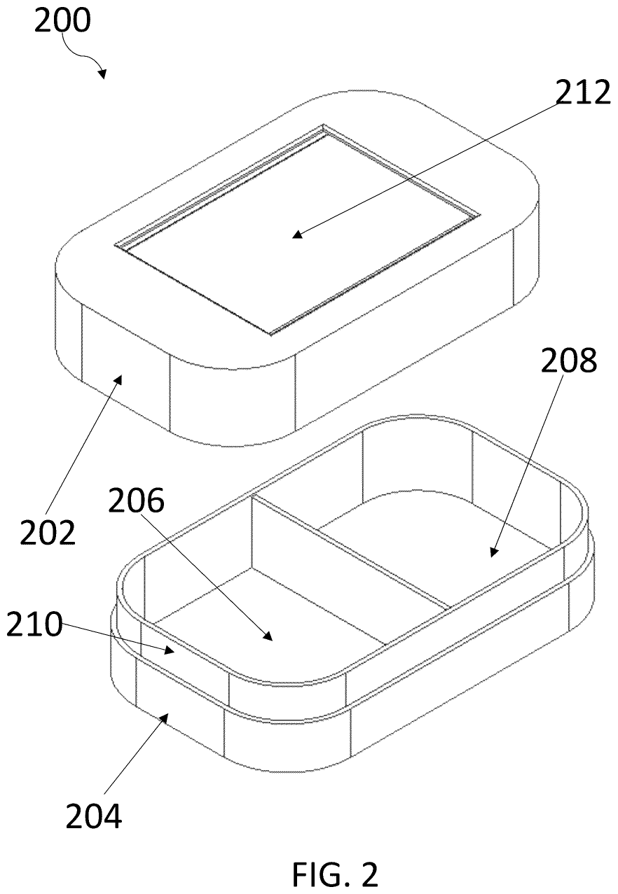

FIG. 2 illustrates an exploded view of an oral consumable tracker device according to at least one aspect of the present disclosure. The oral consumable tracker device 200 includes a top shell 202, a bottom shell 204 including a first compartment 206, a second compartment 208, and an attachment feature 210, and a user interface 212. The oral consumable tracker device can further include at least one power supply component, controllable electronics (e.g., printed circuit board assembly), and means for monitoring use of oral consumables. The oral consumable tracker device can be configured for network communication with at least one remote device. The means for monitoring use of oral consumables can include at least one sensor. The at least one power supply component includes a battery. The battery can be rechargeable or non-rechargeable. Via the user interface, the oral consumable tracker device can receive user input. The user input may include response to survey questions, indication of a start of use of an oral consumable, and indication of an end of use of an oral consumable.

The at least one sensor can include a magnetic field sensor, a linear sensor, a switch sensor, a Hall Effect sensor, a push switch, a proximity sensor, a weight sensor, an image sensor, and/or a motion sensor. The at least one sensor can be positioned on or in the bottom shell and/or the top shell. For further example, in at least one aspect of the present disclosure, the oral consumable tracker device can monitor a change in a weight and/or movement of an oral consumable in each compartment. The means for monitoring use of oral consumables can compare a weight before a user removes an oral consumable to a weight when a user returns the oral consumable and/or the oral consumable packaging. The oral consumable tracker device may generate an alert if the weight does not change or is above or below an expected threshold. If the determined weight is within a predetermined threshold, the oral consumable tracker device can automatically update user information to a remote database.

In at least one aspect of the present disclosure, the oral consumable tracker device includes a controllable layer positioned between the top shell and the bottom shell. In at least one aspect of the present disclosure, the top shell is removably attached to the bottom shell. For example, and without limitation, the bottom shell includes at least one feature for receiving the top shell. The feature can include a channel, a slot, or a similar feature for receiving the top shell. In at least one aspect of the present disclosure, the top shell can nestle in the bottom shell. In at least one aspect of the present disclosure, the top shell can snap onto the bottom shell.

In at least one aspect of the present disclosure, the controllable layer may include a slidable layer that can cover one or more compartments of the at least two compartments. The controllable layer may be manually controlled, controlled by the at least one processor, and/or remotely controlled. For example, and without limitation, the controllable layer may include a slidable door that can be physically moved by a user. In at least one aspect of the present disclosure, the at least one processor can control the position of the controllable layer. For example, and without limitation, the at least one processor can detect that a user is beginning a session (e.g. user input via the user interface), and, in response, move the controllable layer to uncover a compartment that includes an oral consumable. For further example, and without limitation, the at least one processor can detect when at least one consumable is removed from a compartment, and, in response, move the at least one controllable layer to cover the compartment corresponding to the removed oral consumable. Advantageously, this helps improve accuracy during studies by limiting access to oral consumables. In at least one aspect of the present disclosure, the oral consumer trackable device can receive a command from at least one remote device corresponding to the position of the controllable layer.

The processor manages the overall operations of the oral consumable tracker device. The processor is any controller, microcontroller, or microprocessor that is capable of processing program instructions. In at least one aspect of the present disclosure, the oral consumable tracker device includes at least one antenna, which enables the oral consumable tracker device to send information (e.g., oral consumable consumption, battery level) to at least one remote device (e.g., smartphone, tablet, laptop computer) and/or receive information (e.g., user survey questions) from at least one remote device. The at least one antenna provides wireless communication, standards-based or non-standards-based, by way of example and not limitation, radiofrequency (RF), BLUETOOTH, ZIGBEE, NEAR FIELD COMMUNICATION (NFC), or other similar communication methods.

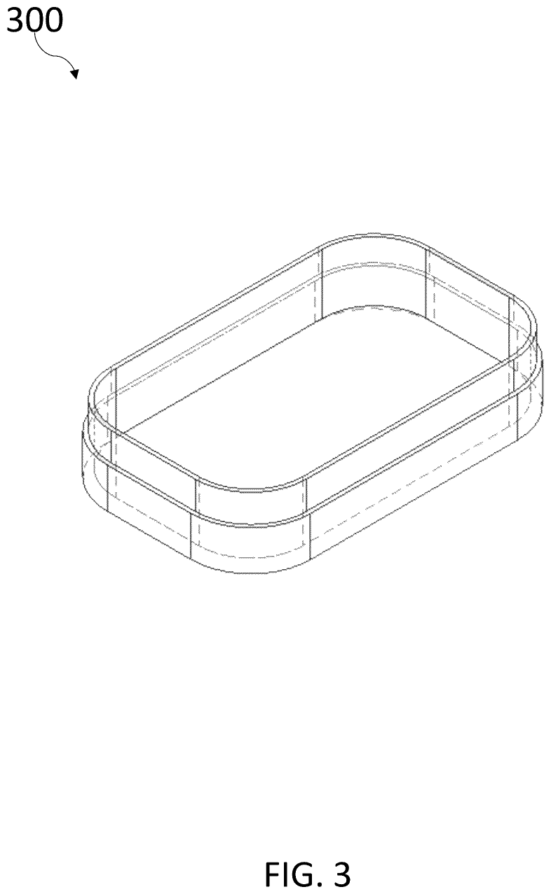

FIG. 3 illustrates a perspective view of a bottom shell of an oral consumable tracker device according to at least one aspect of the present disclosure. The bottom shell can include means for monitoring use of one or more oral consumables. The oral consumable tracker device can further include at least one power supply component and controllable electronics (e.g., printed circuit board assembly). The oral consumable tracker device can be configured for network communication with at least one remote device. The means for monitoring use of oral consumables can include at least one sensor. The at least one power supply component includes a battery. The battery can be rechargeable or non-rechargeable. Via the user interface, the oral consumable tracker device can receive user input. The user input may include response to survey questions, indication of a start of use of an oral consumable, and indication of an end of use of an oral consumable.

The at least one sensor can include a magnetic field sensor, a linear sensor, a switch sensor, a Hall Effect sensor, a push switch, a proximity sensor, a weight sensor, an image sensor, and/or a motion sensor. The at least one sensor can be positioned on or in the bottom shell and/or the top shell. For further example, in at least one aspect of the present disclosure, the oral consumable tracker device can monitor a change in a weight and/or movement of an oral consumable in the compartment. The means for monitoring use of oral consumables can compare a weight before a user removes an oral consumable to a weight when a user returns the oral consumable and/or the oral consumable packaging. The oral consumable tracker device may generate an alert if the weight does not change or is above or below an expected threshold. If the determined weight is within a predetermined threshold, the oral consumable tracker device can automatically update user information to a remote database.

In at least one aspect of the present disclosure, the means for monitoring usage of an oral consumable includes detecting a change in the bottom shell of the oral consumable tracker device. For example, and without limitation, the means for monitoring usage of an oral consumable may include a weight sensor designed to monitor changes in weight of items located on an interior surface of the bottom shell. The means for monitoring usage of an oral consumable may include an image sensor designed for capturing and analyzing images of an interior of a the bottom shell.

In at least one aspect of the present disclosure, the oral consumable tracker device includes a controllable layer positioned between the top shell and the bottom shell. In at least one aspect of the present disclosure, the top shell is removably attached to the bottom shell. For example, and without limitation, the bottom shell includes at least one feature for receiving the top shell. The feature can include a channel, a slot, or a similar feature for receiving the top shell. In at least one aspect of the present disclosure, the top shell can nestle in the bottom shell. In at least one aspect of the present disclosure, the top shell can snap onto the bottom shell.

In at least one aspect of the present disclosure, the controllable layer may include a slidable layer that can cover one or more compartments of the at least two compartments. The controllable layer may be manually controlled, controlled by the at least one processor, and/or remotely controlled. For example, and without limitation, the controllable layer may include a slidable door that can be physically moved by a user. In at least one aspect of the present disclosure, the at least one processor can control the position of the controllable layer. For example, and without limitation, the at least one processor can detect that a user is beginning a session (e.g. user input via the user interface), and, in response, move the controllable layer to uncover the compartment. For further example, and without limitation, the at least one processor can detect when at least one consumable is removed from the compartment, and, in response, move the at least one controllable layer to cover the compartment. Advantageously, this helps improve accuracy during studies by limiting access to oral consumables. In at least one aspect of the present disclosure, the oral consumer trackable device can receive a command from at least one remote device corresponding to the position of the controllable layer.

The processor manages the overall operations of the oral consumable tracker device. The processor is any controller, microcontroller, or microprocessor that is capable of processing program instructions. In one embodiment, the control electronics includes at least one antenna, which enables the oral consumable tracker device to send information (e.g., oral consumable consumption, battery level) to at least one remote device (e.g., smartphone, tablet, laptop computer) and/or receive information (e.g., user survey questions) from at least one remote device. The at least one antenna provides wireless communication, standards-based or non-standards-based, by way of example and not limitation, radiofrequency (RF), BLUETOOTH, ZIGBEE, NEAR FIELD COMMUNICATION (NFC), or other similar communication methods.

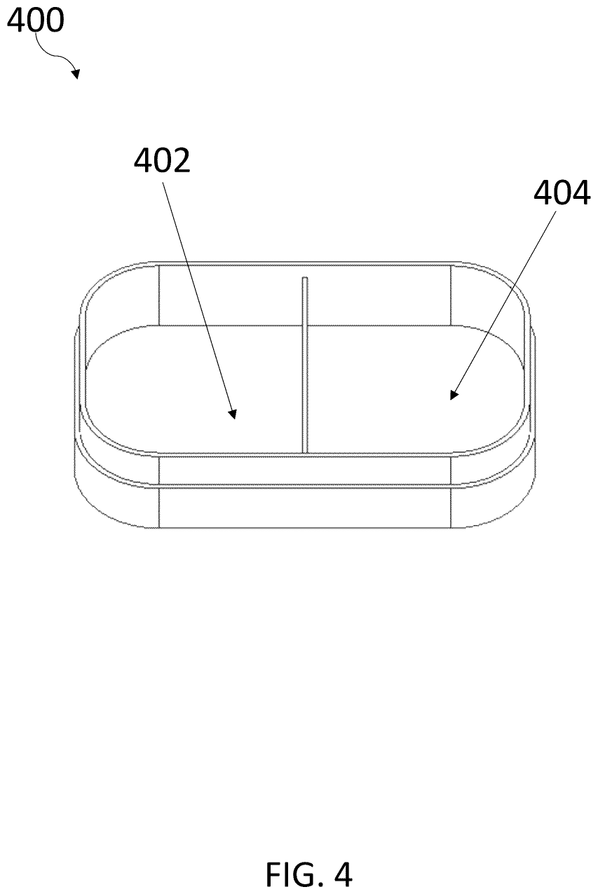

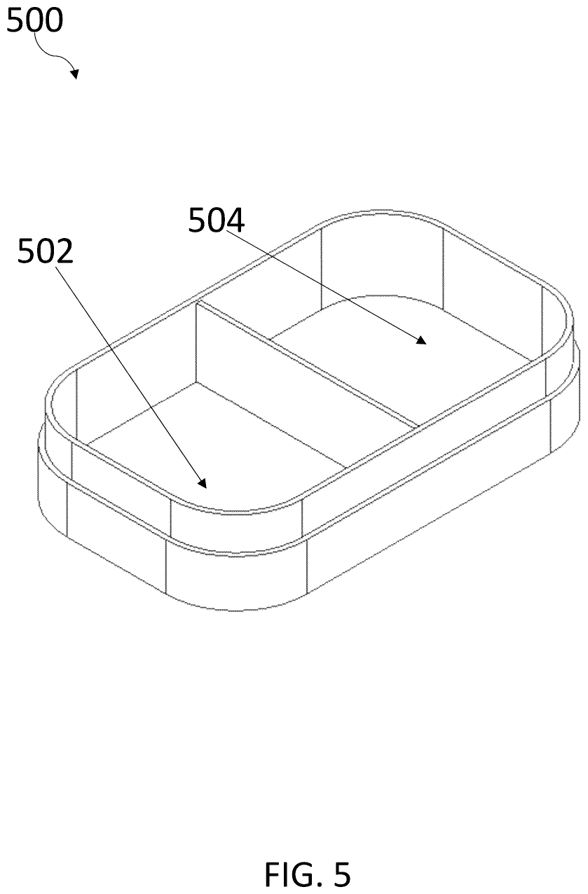

FIG. 4 illustrates a perspective view of a bottom shell of an oral consumable tracker device with two compartments according to at least one aspect of the present disclosure. The bottom shell 400 of the oral consumable tracker device includes a first compartment 402 and a second compartment 404. Each compartment can include means for tracking use of one or more oral consumables. FIG. 5 illustrates a perspective view of a bottom shell 500 of an oral consumable tracker device according to at least one aspect of the present disclosure. The bottom shell 500 of the oral consumable tracker device includes a first compartment 502 and a second compartment 504. Each compartment can include means for tracking use of one or more oral consumables.

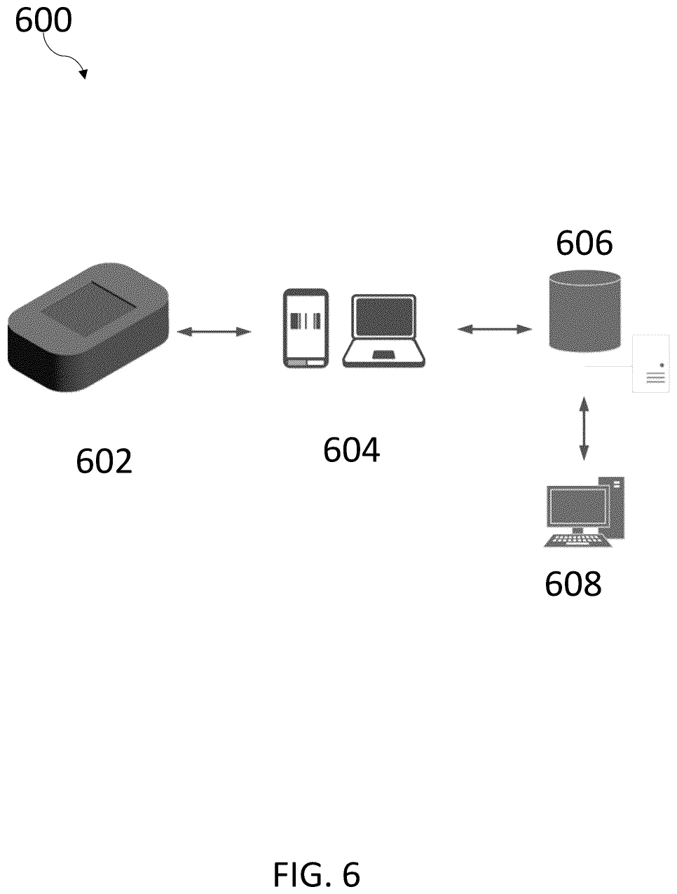

FIG. 6 illustrates a schematic diagram of an oral consumable tracker device system according to at least one aspect of the present disclosure. The oral consumable tracker device system 600 includes an oral tracker device 602 in network communication with at least one remote device 604, at least one database 606, and at least one remote server 608. The oral tracker device 602 is configured to track use of one or more oral consumables. The oral tracker device 602 can receive user input corresponding to consumption and usage of the oral consumables. For example, and without limitation, the oral tracker device 602 can receive user input corresponding to the beginning of a session, ending of a session, and answer to prompts corresponding to the user activity. The oral tracker device can transmit the user input and user activity data to the at least one remote device 604. The user input data and user activity data can be transmitted to the at least one database 606 and/or the at least one remote server 608. The at least one remote device and/or the at least one remote server can analyze the user input and user activity data to determine adherence with a research study.

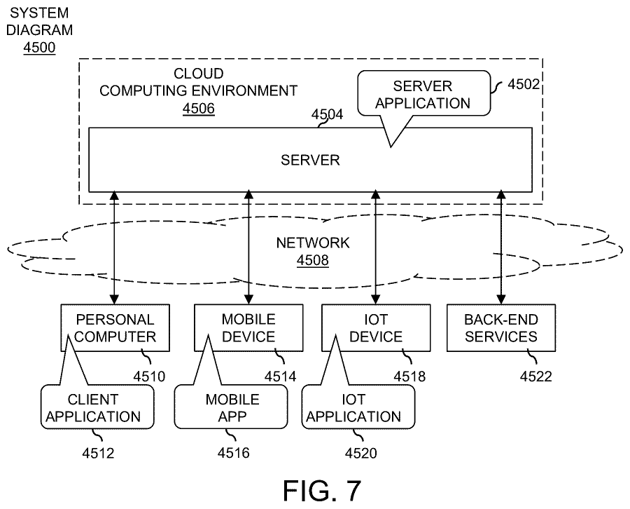

FIG. 7 depicts a system diagram 4500 illustrating a client/server architecture in accordance with embodiments of the present disclosure. The server application 4502 is configured to provide a video application and mobile application for an oral consumable tracking system. A server application 4502 is hosted on a remote server 4504 within a cloud computing environment 4506. The server application 4502 is provided on a non-transitory computer-readable medium including a plurality of machine-readable instructions, which when executed by one or more processors of the server 4504, are adapted to cause the server 4504 to generate the video platform and mobile application.

The server application 4502 is configured to communicate over a network 4508. In a preferred embodiment, the network 4508 is the Internet. In other embodiments, the network 4508 may be restricted to a private local area network (LAN) and/or private wide area network (WAN). The network 4508 provides connectivity with a plurality of client devices including a personal computer 4510 hosting a client application 4512, and a mobile device 4514 hosting a mobile app 4516. The network 4508 also provides connectivity for an Internet-Of-Things (IoT) device 4518 hosting an IoT application 4520, to back-end services 4522. Advantageously, the back-end services are operable to communicate with third-party application programming interfaces (APIs) to either provide or receive data that can be used by the system to provide recommendations. Third-party applications can provide algorithms for analysis of data. The back-end services may provide data gathered within the oral consumable tracking system through the third-party APIs and receive results from the algorithms provided back to the back-end services to provide further recommendations or take further actions within the oral consumable tracking system.

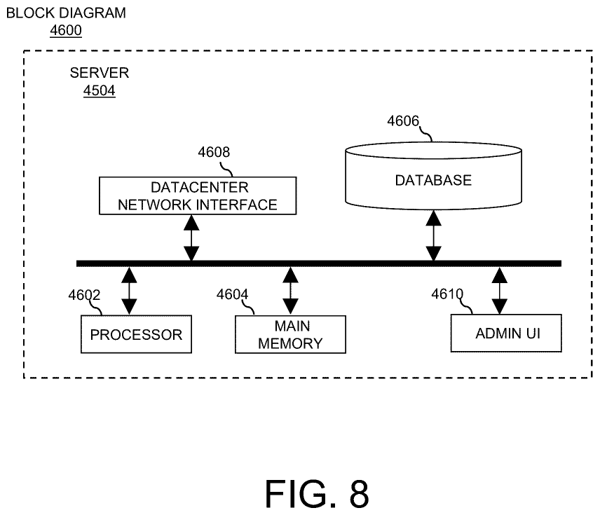

FIG. 8 depicts a block diagram 4600 of the server 4504 of FIG. 7 for hosting at least a portion of the server application 4502 of FIG. 7 in accordance with embodiments of the present disclosure. The server 4504 may be any of the hardware servers referenced in this disclosure. The server 4504 may include at least one of a processor 4602, a main memory 4604, a database 4606, a datacenter network interface 4608, and an administration user interface (UI) 4610. The server 4504 may be configured to host one or more virtualized servers. For example, the virtual server may be an Ubuntu® server or the like. The server 4504 may also be configured to host a virtual container. For example, the virtual server may be the DOCKER® virtual server or the like. In some embodiments, the virtual server and or virtual container may be distributed over a plurality of hardware servers using hypervisor technology.

The processor 4602 may be a multi-core server class processor suitable for hardware virtualization. The processor 4602 may support at least a 64-bit architecture and a single instruction multiple data (SIMD) instruction set. The memory 4604 may include a combination of volatile memory (e.g., random access memory) and non-volatile memory (e.g., flash memory). The database 4606 may include one or more hard drives.

The datacenter network interface 4608 may provide one or more high-speed communication ports to the data center switches, routers, and/or network storage appliances. The datacenter network interface may include high-speed optical Ethernet, InfiniBand (IB), Internet Small Computer System Interface iSCSI, and/or Fibre Channel interfaces. The administration UI may support local and/or remote configuration of the server by a data center administrator.

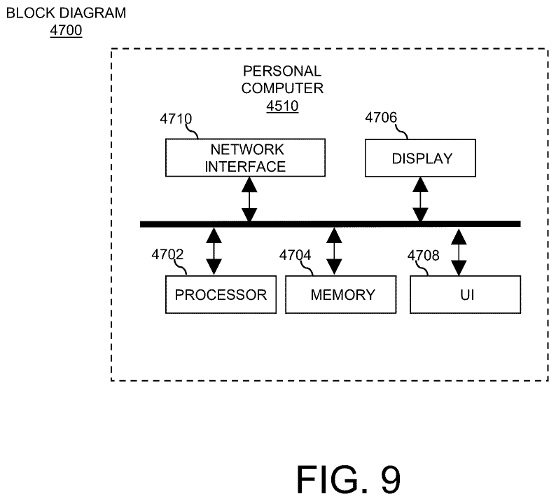

FIG. 9 depicts a block diagram 4700 of the personal computer 4510 of FIG. 7 in accordance with embodiments of the present disclosure. The personal computer 4510 may be any of the devices referenced in this disclosure. The personal computer 4510 may include at least a processor 4702, a memory 4704, a display 4706, a user interface (UI) 4708, and a network interface 4710. The personal computer 4510 may include an operating system to run a web browser and/or the client application 4512 shown in FIG. 7. The operating system (OS) may be a Windows® OS, a Macintosh® OS, or a Linux® OS. The memory 4704 may include a combination of volatile memory (e.g., random access memory) and non-volatile memory (e.g., solid state drive and/or hard drives).

The network interface 4710 may be a wired Ethernet interface or a Wi-Fi interface. The personal computer 4510 may be configured to access remote memory (e.g., network storage and/or cloud storage) via the network interface 4710. The UI 4708 may include a keyboard, and a pointing device (e.g., mouse). The display 4706 may be an external display (e.g., computer monitor) or internal display (e.g., laptop). In some embodiments, the personal computer 4510 may be a smart TV. In other embodiments, the display 4706 may include a holographic projector.

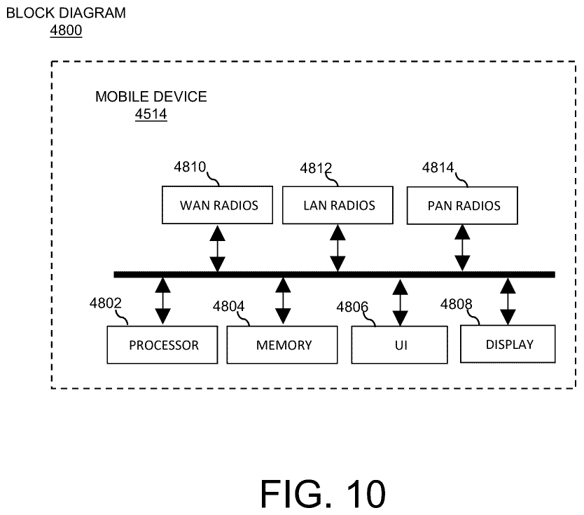

FIG. 10 depicts a block diagram 4800 of the mobile device 4514 of FIG. 7 in accordance with embodiments of the present disclosure. The mobile device 4514 may be any of the remote devices referenced in this disclosure. The mobile device 4514 may include an operating system to run a web browser and/or the mobile app 4516 shown in FIG. 7. The mobile device 4514 may include at least a processor 4802, a memory 4804, a UI 4806, a display 4808, WAN radios 4810, LAN radios 4812, and personal area network (PAN) radios 4814. In some embodiments the mobile device 4514 may be an iPhone® or an iPad®, using iOS® as an OS. In other embodiments, the mobile device 4514 may be a mobile terminal including Android® OS, BlackBerry® OS, Chrome® OS, Windows Phone® OS, or the like.

In some embodiments, the processor 4802 may be a mobile processor such as the Qualcomm® Snapdragon™ mobile processor. The memory 4804 may include a combination of volatile memory (e.g., random access memory) and non-volatile memory (e.g., flash memory). The memory 4804 may be partially integrated with the processor 4802. The UI 4806 and display 4808 may be integrated such as a touchpad display. The WAN radios 4810 may include 2G, 3G, 4G, and/or 5G technologies. The LAN radios 4812 may include Wi-Fi technologies such as 802.11a, 802.11b/g/n, and/or 802.11ac circuitry. The PAN radios 4814 may include Bluetooth® technologies.

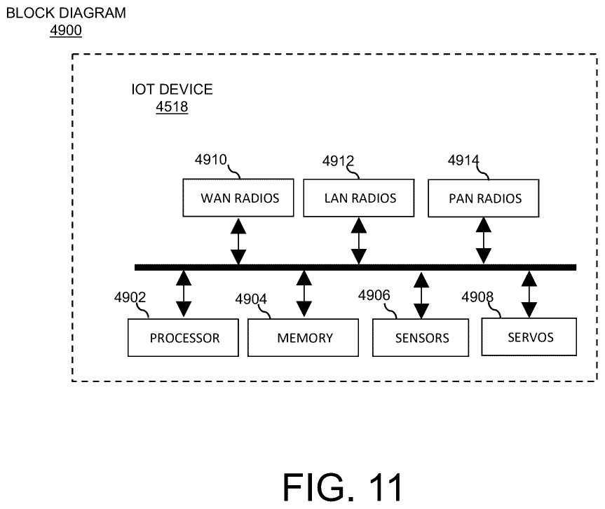

FIG. 11 depicts a block diagram 4900 of the IoT device 4518 of FIG. 7 in accordance with embodiments of the present disclosure. The IoT device 4518 may be any of the remote devices referenced in this disclosure. The IoT device 4518 includes a processor 4902, a memory 4904, sensors 4906, servos 4908, WAN radios 4910, LAN radios 4912, and PAN radios 4914. The processor 4902, a memory 4904, WAN radios 4910, LAN radios 4912, and PAN radios 4914 may be of similar design to the processor 4802, a memory 4804, WAN radios 4810, LAN radios 4812, and PAN radios 4814 of the mobile device 4514 of FIG. 7. The sensors 4906 and servos 4908 may include any applicable components related to IoT devices such as a monitoring device, an autonomous vehicle, a home assistant, a smart appliance, a medical device, a virtual reality device, an augmented reality device, or the like.

Any combination of one or more computer-readable medium(s) may be utilized. The computer-readable medium may be a computer readable signal medium or a computer-readable storage medium (including, but not limited to, non-transitory computer-readable storage media). A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer readable storage medium and that can communicate, propagate, or transport a program for use by or in connection with an instruction execution system, apparatus, or device.

In at least one aspect of the present disclosure, the oral consumable tracking system includes a cloud-based network for distributed communication via a wireless communication antenna and processing by at least one mobile communication computing device. In another embodiment of the present disclosure, the system is a virtualized computing system capable of executing any or all aspects of software and/or application components presented herein on computing devices. In certain aspects, the computer system may be implemented using hardware or a combination of software and hardware, either in a dedicated computing device, or integrated into another entity, or distributed across multiple entities or computing devices.

By way of example, and not limitation, the computing devices are intended to represent various forms of digital computers and mobile devices, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the disclosure described and/or claimed in this document.

In one embodiment, the computing device includes components such as a processor, a system memory having a random-access memory (RAM) and a read-only memory (ROM), and a system bus that couples the memory to the processor. In another embodiment, the computing device may additionally include components such as a storage device for storing the operating system and one or more application programs, a network interface unit, and/or an input/output controller. Each of the components may be coupled to each other through at least one bus. The input/output controller may receive and process input from, or provide output to, a number of other devices, including, but not limited to, alphanumeric input devices, mice, electronic styluses, display units, touch screens, signal generation devices (e.g., speakers), or printers.

By way of example, and not limitation, the processor may be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another embodiment, multiple processors and/or multiple buses may be used, as appropriate, along with multiple memories of multiple types (e.g., a combination of a DSP and a microprocessor, a plurality of microprocessors, one or more microprocessors in conjunction with a DSP core).

Also, multiple computing devices may be connected, with each device providing portions of the necessary operations (e.g., a server bank, a group of blade servers, or a multi-processor system). Alternatively, some steps or methods may be performed by circuitry that is specific to a given function. According to various embodiments, the computer system may operate in a networked environment using logical connections to local and/or remote computing devices through a network. A computing device may connect to a network through a network interface unit connected to a bus. Computing devices may communicate communication media through wired networks, direct-wired connections or wirelessly, such as acoustic, RF, or infrared, through an antenna in communication with the network antenna and the network interface unit, which may include digital signal processing circuitry when necessary. The network interface unit may provide for communications under various modes or protocols.

In one or more exemplary aspects, the instructions may be implemented in hardware, software, firmware, or any combinations thereof. A computer readable medium may provide volatile or non-volatile storage for one or more sets of instructions, such as operating systems, data structures, program modules, applications, or other data embodying any one or more of the methodologies or functions described herein. The computer readable medium may include the memory, the processor, and/or the storage media and may be a single medium or multiple media (e.g., a centralized or distributed computer system) that store the one or more sets of instructions. Non-transitory computer readable media includes all computer readable media, with the sole exception being a transitory, propagating signal per se. The instructions may further be transmitted or received over the network via the network interface unit as communication media, which may include a modulated data signal such as a carrier wave or other transport mechanism and includes any delivery media. The term “modulated data signal” means a signal that has one or more of its characteristics changed or set in a manner as to encode information in the signal.

Storage devices and memory include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system.

The various illustrative logical blocks, modules, elements, circuits, and algorithms described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present disclosure.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A device for tracking consumption of an oral consumable, the device comprising:
a housing including a top shell and a bottom shell;
at least one processor;
at least one antenna; and
a user interface;
wherein the bottom shell includes at least two compartments, wherein each compartment of the at least two compartments include means for monitoring oral consumables; and
wherein the means for monitoring oral consumables is configured to detect and monitor one or more oral consumables positioned inside the housing.

2. The device of claim 1, wherein the means for monitoring oral consumables includes at least one sensor, wherein the at least one sensor includes a hall effect sensor, a proximity sensor, or a push switch.

3. The device of claim 1 further configured to display user prompts via the user interface.

4. The device of claim 1, wherein the at least two compartments further comprise a first compartment and a second compartment.

5. The device of claim 1, further comprising at least one slidable component positioned between the top shell and the bottom shell, wherein the at least one slidable component is configured to cover at least one compartment of the at least two compartments.

6. The device of claim 5, wherein the at least one processor is configured to control a position of the at least one slidable component.

7. The device of claim 6, wherein the at least one processor to receive user input that a user is beginning a session, wherein, in response, the at least one processor is configured to uncover a first compartment to provide access to at least one oral consumable.

8. The device of claim 7, wherein the at least one processor is configured to detect when at least one consumable has been removed from the first compartment, wherein the at least one processor is configured to activate the at least one slidable component to cover the first compartment.

9. A device for tracking consumption of an oral consumable, the device comprising:
a housing including a top shell, a bottom shell, and a slidable cover;
at least one processor;
at least one antenna; and
a user interface;
wherein the bottom shell includes at least two compartments, wherein each compartment of the at least two compartments include means for monitoring oral consumables;
wherein the slidable cover is configured to cover at least one compartment of the at least two compartments; and
wherein the means for monitoring oral consumables is configured to detect and monitor one or more oral consumables positioned inside the housing.

10. The device of claim 9, wherein the slidable cover is positioned between the top shell and the bottom shell.

11. The device of claim 10, wherein the at least one processor is configured to control a position of the at least one slidable cover.

12. The device of claim 11, wherein the at least one processor to receive user input that a user is beginning a session, wherein, in response, the at least one processor is configured to uncover a first compartment to provide access to at least one oral consumable.

13. The device of claim 12, wherein the at least one processor is configured to detect when at least one consumable has been removed from the first compartment, wherein the at least one processor is configured to activate the at least one slidable cover to cover the first compartment.

14. The device of claim 9, wherein the means for monitoring oral consumables includes at least one sensor, wherein the at least one sensor is configured to detect a change corresponding to at least one oral consumable positioned in at least one compartment of the at least two compartments.

15. The device of claim 9, wherein the means for monitoring oral consumables includes at least one of a hall effect sensor, a proximity sensor, or a push switch.

16. A device for tracking consumption of an oral consumable, the device comprising:
a housing including a top shell and a bottom shell;
at least one processor;
at least one antenna;
a user interface; and
means for monitoring oral consumables;
wherein the top shell is removably attached to the bottom shell;
wherein the means for monitoring oral consumables is configured to detect and monitor one or more oral consumables positioned inside the housing.

17. The device of claim 16, wherein the means for monitoring oral consumables includes at least one sensor.

18. The device of claim 17, wherein the at least one sensor includes a hall effect sensor, a proximity sensor, or a push switch.

19. The device of claim 16 further configured for network communication with at least one remote device, wherein, based on detection of a change of at least one oral consumable positioned in the housing, the device is configured to transmit updated oral consumable data to the at least one remote device.

20. The device of claim 16 further comprising at least one controllable layer, wherein the controllable layer is positioned between the top shell and the bottom shell, wherein, based on user input received via the user interface, the at least one processor is configured to change a position of the controllable layer.
